(19) United States
(12) Patent Application Publication
(10) Pub. No.: US 2002/0132803 A1
(43) Pub. Date: Sep. 19, 2002

(54) Fluticasone suspension formulation, spray pattern method, and nasal spray apparatus

(76) Inventors: Mahendra Dedhiya, Dublin, OH (US); Julia J. Economou, Hilliard, OH (US)

Correspondence Address: BOEHRINGER INGELHEIM CORPORATION, 900 RIDGEBURY ROAD, P O BOX 368, RIDGEFIELD, CT, 06877, US

(21) Appl. No.: 09/754,777

(22) Filed: Jan. 5, 2001

(51) Int. Cl. A61K 31/56

(52) U.S. Cl. 514/182

(57) ABSTRACT

An aqueous pharmaceutical formulation suitable for use in a pump spray device, comprising (a) fluticasone propionate, (b) an antimicrobial preservative, (c) a surfactant, (d) a tonicity agent, and (e) a suspending agent; methods for using the aqueous pharmaceutical formulation, and suitable pump spray devices.

### BACKGROUND OF THE INVENTION

Fluticasone propionate is the approved name for S-fluoromethyl-6a,9a-difluoro-11b -hydroxy-16a-methyl-17a-propionyloxy-3-oxandrosta-1,4-diene- 1 7b-carbothioate, a corticosteroid known to exhibit topical antiinflammatory activity and described and claimed in Phillipps et al., U.S. Pat. No. 4,335,121. Fluticasone propionate is indicated for the management of the nasal symptoms of seasonal and perennial allergic and nonallergic rhinitis in adults and pediatric patients. In addition, in the treatment of asthmatic conditions, it has been found to be effective to administer fluticasone propionate in the form of dry powders or aerosols containing small particles of the medicament, conventionally prepared by micronization. Conventionally, fluticasone propionate nasal spray has been administered by means of a metered dose nasal spray device and aerosols have been administered by means of metered dose inhalers, which are designed to deliver a fixed unit dosage of medicament per actuation or “puff”.

The spray pattern test is now a widely accepted in vitro test for nasal pump spray delivery systems for all such nasally-delivered pharmaceutical formulations. The spray pattern test was developed to assure equivalent drug deposition patterns, resulting in equivalent delivery of the drug, for example, fluticasone propionate, to nasal site of action and equivalent systemic exposure or absorption. The spray pattern test also assists in establishing in vitro bioavailability of the product in accordance, for example, with the publication of the U.S. Food and Drug Administration's (FDA) Draft Guidance for Industry: Bioavailability and Bioequivalence Studies for Nasal Aerosols and Nasal Spray for Local Action (Jun. 1999)(incorporated herein by reference in its entirety and hereinafter referred to as “FDA Draft Guidance”). In order to meet the FDA Draft Guidance, which may become mandatory for the approval of such products, it has therefore become important to administer nasal sprays in a controlled manner, such that the nasal spray produced has a desired and reproducible shape.

### BRIEF SUMMARY OF THE INVENTION

The invention includes a suspension formulation containing fluticasone propionate in a pharmaceutically acceptable vehicle for nasal administration. In one aspect of the present invention there is provided an aqueous pharmaceutical formulation suitable for use in a pump spray device, comprising:

(a) fluticasone propionate;

(b) an antimicrobial preservative;

(c) a surfactant;

(d) a tonicity agent; and

(e) a suspending agent.

In a preferred embodiment of the invention, the antimicrobial preservative is selected from the group consisting of: benzalkonium chloride, methylparaben, sodium benzoate, benzoic acid, phenyl ethyl alcohol, mixtures thereof, and the like. In another preferred embodiment of the invention, the surfactant is selected from the group consisting of: Polysorbate 80 NF, polyoxyethylene 20 sorbitan monolaurate, polyoxyethylene (4) sorbitan monolaurate, polyoxyethylene 20 sorbitan monopalmitate, polyoxyethylene 20 sorbitan monostearate, polyoxyethylene (4) sorbitan monostearate, polyoxyethylene 20 sorbitan tristearate, polyoxyethylene (5) sorbitan monooleate, polyoxyethylene 20 sorbitan trioleate, polyoxyethylene 20 sorbitan monoisostearate, sorbitan monooleate, sorbitan monolaurate, sorbitan monopalmitate, sorbitan monostearate, sorbitan trilaurate, sorbitan trioleate, sorbitan tristearate, mixtures thereof, and the like. In yet another preferred embodiment of the invention, the tonicity agent is selected from the group consisting of: dextrose, lactose, sodium chloride, mixtures thereof, and the like. In a further preferred embodiment of the invention, the suspending agent is selected from the group consisting of: microcrystalline cellulose, carboxymethylcellulose sodium NF, polyacrylic acid, magnesium aluminum silicate, xanthan gum, mixtures thereof, and the like.

In preferred embodiments of the present invention, the aqueous pharmaceutical formulation comprises about 0.03% to about 0.07% (w/w), more preferably about 0.04% to about 0.06% (w/w), and most preferably about 0.04% to about 0.05% (w/w) of fluticasone propionate. In other preferred embodiments of the present invention, the aqueous pharmaceutical formulation comprises about 0.01% to about 0.50% (w/w), more preferably about 0.08% to about 0.40% (w/w), and most preferably about 0.10% to about 0.30% (w/w), of the antimicrobial preservative. In yet other preferred embodiments of the present invention, the aqueous pharmaceutical formulation comprises about 0.001% to about 0.050% (w/w), more preferably about 0.004% to about 0.030% (w/w), and most preferably about 0.005% to about 0.020% (w/w) of the surfactant. In preferred embodiments of the present invention, the aqueous pharmaceutical formulation comprises about 1.0% to about 10.0% (w/w), more preferably about 3.0% to about 7.0% (w/w), and most preferably about 4.0% to about 6.0% (w/w) of the tonicity agent. In yet other preferred embodiments of the present invention, the aqueous pharmaceutical formulation comprises about 0.5% to about 5.0% (w/w), more preferably about 1.0% to about 3.0% (w/w), and most preferably about 1.5% to about 2.0% (w/w) of the suspending agent.

The invention also comprises a method of administering a pharmaceutical formulation, such as the novel aqueous pharmaceutical formulation that set forth above, to produce a spray pattern having desired characteristics as measured by ovality of the spray pattern. Thus, in another aspect of the present invention there is provided a method of administering a pharmaceutical formulation, for example, the novel aqueous pharmaceutical formulation according to the invention comprising fluticasone, to a host in need of such treatment, comprising spraying the pharmaceutical formulation using a nasal pump spray device, wherein the average ovality ratio of the spray produced is between about 1.0 and about 1.7, more preferably between about 1.1 and about 1.5, even more preferably between about 1.2 and about 1.4, and most preferably between about 1.2 and about 1.3.

The invention also comprises a pharmaceutically acceptable nasal spray device including a swirl chamber dimensioned to produce a spray pattern having the desired average ovality ratio. This pharmaceutically acceptable nasal spray device may be used with the novel aqueous pharmaceutical formulation that set forth above or other pharmaceutical formulations. Therefore, in another aspect of the present invention there is provided a pump spray device for a pharmaceutical formulation, the pump spray device comprising an actuator and a pump, wherein the improvement comprises: a swirl chamber insert and a central swirl chamber with at least three channels, each channel extending from the center of the swirl chamber to the outer diameter of the swirl chamber, wherein the ratio of the diameter of the center of the swirl chamber to the average width of the channels is between about 2.5 and about 3.3, more preferably between about 2.6 and about 3.1, even more preferably between about 2.7 and about 3.0, and most preferably between about 2.8 and about 3.0.

The invention also comprises fluticasone propionate having a defined surface area for appropriate local action. Thus, in another aspect of the present invention there is provided fluticasone propionate having a surface area (BET) in the range of about 7 m²/g to about 12 m²/g, more preferably in the range of about 8 m²/g to 10 m²/g, and most preferably in the range of about 9 m²/g to 10 m²/g.

### BRIEF DESCRIPTION OF THE FIGURES

FIG. 1 is a plan drawing of the pump portion of a conventional pump spray device used to illustrate the present invention with the various parts indicated.

FIG. 2 is a plan drawing of an actuator portion of a conventional pump spray device used to illustrate the present invention with the various parts indicated.

FIG. 3 is a diagram showing a central swirl chamber for use with the pump spray device according to the present invention with the various dimension measurements indicated.

FIG. 4 shows the spray patterns obtained on a TLC plate for a pump spray device according to the present invention for single sprays of an aqueous fluticasone propionate formulation according to the present invention at various distances measured from the actuator to the TLC plate surface.

### DEFINITIONS OF TERMS

In describing the invention the following terms as used herein have been defined to better describe the invention in all its aspects.

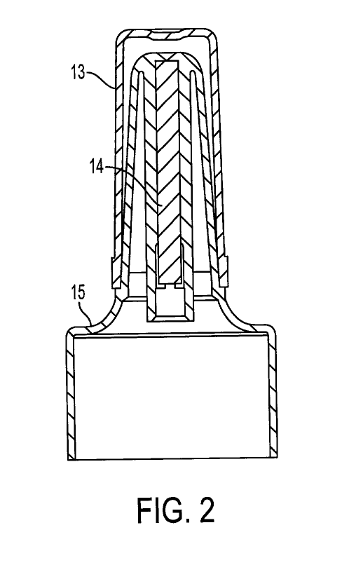

The term “swirl chamber insert” designates the part in nasal pump spray device that receives the metered dose of the pharmaceutical formulation from the pump portion of the nasal pump spray device and directs the metered dose into the central swirl chamber. An example of the swirl chamber insert is shown in FIG. 2.

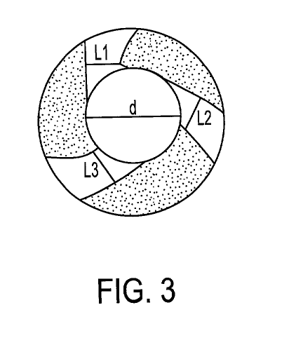

The term “central swirl chamber” means the chamber at the top portion of the swirl chamber insert that receives the metered dose of the pharmaceutical formulation from the swirl chamber insert. The diameter of the central swirl chamber is the diameter d of the inner circular portion of the central swirl chamber, for example, illustrated in FIG. 3. The term “center of the swirl chamber” is the center of the swirl chamber, which is the location of the orifice (not shown) through which the pharmaceutical formulation exits the nasal pump spray device.

The terms “swirl chamber channels” or “channels” means the channels defined by the two channel walls and extending from the central swirl chamber to the outer diameter of the swirl chamber insert, which direct the metered dose of pharmaceutical formulation in a spray pattern external to the nasal pump spray device. The width of the channels is the width of the channels measured at a line perpendicular to the longer channel wall to the shorter channel wall nearest the central swirl chamber, illustrated as L1, L2, and L3 in FIG. 3.

The term “ovality ratio” as used herein is defined according to the FDA Draft Guidance and means the ratio of the widest (Dmax) and shortest (Dmin) diameters of a spray pattern following impaction on an appropriate target upon a single actuation of fluticasone propionate nasal product at a selected distance from the actuator to the target. The ovality ratio provides information about the shape and density of the plume of Fluticasone propionate nasal product following actuation. The ovality ratio is determined at an appropriate selected distance from the pump spray device actuator tip to the target (generally a TLC plate). Typical selected distances, for example, are about 0.5 cm to about 6.0 cm, about 0.75 cm to about 5.0 cm, about 1.0 cm to about 4.0 cm, about 1.5 cm to about 3.5 cm, and about 2.0 cm to about 3.0 cm.

The term “average ovality ratio” as used herein is the average ovality ratio produced from a pump spray device as determined from 3 or more consecutive sprays at 3 different distances between 0.5 to 6.0 cm.

The term “surface area (BET)” as used herein has the meaning defined in the U.S. Pharmacopoeia (USP 24) and is the specific surface area of a powder determined by physical adsorption of a gas on the surface of the fluticasone propionate solid and by measuring the amount of adsorbate gas corresponding to a single layer on the surface.

The term “average particle size” as used herein means a statistical mean value of the drug particle measured from at least 3 consecutive measurements.

As used herein, the term “drug” means any nasally-administered pharmaceutically effective compound, including anti-inflammatory drugs and systemically effective drugs. The term “drug” is intended to include both presently available pharmaceutically active drugs used therapeutically and therapeutically effective drugs that will be developed in the future that can be nasally administered for local exposure or systemic absorption.

The term “anti-inflammatory drug” as used herein means any pharmaceutically effective compound used in the treatment of any inflammatory disease and, in particular, the treatment of diseases related to seasonal and perennial allergic and nonallergic rhinitis. Such anti-inflammatory drugs include those which are listed within the Physicians' Desk Reference, ₅₄th Edition (2000), incorporated herein by reference in its entirety, including steroids such as beclomethasone dipropionate, flunisolide, fluticasone, budesonide, mometasone, and triamcinolone acetonide, particularly fluticasone. Other anti-inflammatory drugs include cromoglycates such as cromolyn sodium.

The terms “pharmaceutical formulation”, “suspension formulation”, and “aqueous pharmaceutical formulation” and the like are used herein to describe a pharmaceutically active drug or anti-inflammatory drug by itself or with a pharmaceutically acceptable carrier in flowable liquid or suspension form. Such formulations are preferably solutions and suspension, e.g., aqueous suspension and solutions, ethanolic suspension and solutions, aqueous/ethanolic suspension and solutions, saline solutions, and colloidal suspensions.

In general, the drugs and anti-inflammatory drugs are intended to encompass the free acids, free bases, salts, amines, and various hydrate forms including semi-hydrate forms of such drugs and anti-inflammatory drugs. When using the pump spray device according to the present invention or performing the method of administering a pharmaceutical formulation to a host in need of such treatment according to the present invention, it should be understood that the drugs and anti-inflammatory drugs are generally administered in the form of pharmaceutically acceptable formulations of such drugs which are formulated in combination with pharmaceutically acceptable excipient materials generally known to those skilled in the art and such pharmaceutical formulations consist essentially of the drug in combination with pharmaceutically acceptable excipients in a suitable carrier (e.g., water and/or ethanol). It should be noted, however, if a drug is a liquid without an excipient, the formulation may consist solely of a drug that has a sufficiently low viscosity.

### DETAILED DESCRIPTION OF THE INVENTION

One aspect of the present invention comprises a pharmaceutically acceptable nasal spray device including a swirl chamber dimensioned to produce a spray pattern having the desired average ovality ratio. Therefore, in another aspect of the present invention there is provided a pump spray device for a pharmaceutical formulation, the pump spray device comprising an actuator and a pump, wherein the improvement comprises: a swirl chamber insert having a central swirl chamber and at least three channels, each channel extending from the central swirl chamber to the outer diameter of the swirl chamber insert, wherein the ratio of the diameter of the central swirl chamber to the average width of the channels is between about 2.5 and about 3.3, more preferably between about 2.6 and about 3.1, even more preferably between about 2.7 and about 3.0, and most preferably between about 2.8 and about 3.0.

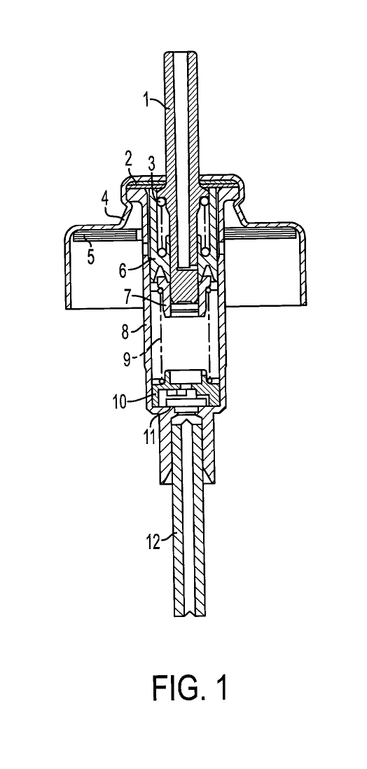

FIG. 1 is a plan drawing of the pump portion of a pump spray device according to the present invention with the various parts indicated. The parts of the pump portion include stem 1, stem gasket 2, stem spring 3, ferrule 4, sealing gasket 5, piston 6, spring cap 7, pump body 8, return spring 9, spring support 10, floating gasket 11, and dip tube 12.

FIG. 2 is a plan drawing of an actuator portion of a conventional pump spray device used to illustrate according to the present invention with the various parts indicated. The actuator includes cap 13, swirl chamber insert 14, and actuator body 15. Actuator body 15 fits over stem 1 of the pump portion of a pump spray device to assemble the device for use.

FIG. 3 is a diagram showing a central swirl chamber according to the present invention for use with the pump spray device with the various dimension measurements indicated. In FIG. 3, symbol d indicates the diameter of the central swirl chamber and the symbols L1, L2, and L3 indicate the width of the channels. At the center of the swirl chamber (i.e., at the midpoint of d) there is the orifice (not shown) from which the pharmaceutical formulation emerges from the nasal pump spray device. During use, the pump spray device functions as a conventional pump. Dip tube 12 is in contact with the liquid pharmaceutical formulation held in a container (not shown) that is sealed by ferrule 4. Generally, the actuator portion must be pressed down by the operator three or more times to prime the pump at the initial operation of the pump spray device. Before pressing down on actuator body 15, cap 13 is removed. At rest, the pump spray device is sealed at stem gasket 2 and sealing gasket 5. When the pump spray device is pressurized by pressing down on actuator body 15, floating gasket 11 is pressed against the base to form a tight seal, which leads to an increase in pressure in the chamber within pump body 8. As the pharmaceutical formulation within pump body 8 cannot be compressed, piston 6 is blocked in its downward movement. Continuing movement of actuator body 15 downward forces stem 1 downward until the orifice near the base of stem 1 is exposed to the pressurized chamber within pump body 8 and the pharmaceutical formulation travels up the central passage within stem 1 through swirl chamber insert 14 to the central swirl chamber according to the present invention at the far end (i.e., farthest from stem 1) of swirl chamber insert 14. The pharmaceutical formulation emerges from swirl chamber insert 14 into the central swirl chamber and is directed along the channels and propelled through the orifice of the central swirl chamber (i.e., at midpoint d) to produce a spray pattern of a desired ovality. At this point spraying is completed and stem spring 3 returns piston 6 to its original position and return spring 9 pushes all moving parts to their original position. While the pump device is returned to its original position, the orifice near the base of stem 1 is isolated from the chamber within pump body 8, creating a vacuum, raising floating gasket 11 and allowing the liquid pharmaceutical formulation held in a container to travel up dip tube 12, repriming the pump spray device with a metered dose of the liquid pharmaceutical formulation.

All of the parts of the pump spray device are made from pharmaceutically acceptable materials appropriate for the pharmaceutical formulation used therein.

To the extent that a pump spray device does not have a central swirl chamber according to the present invention, that is, a central swirl chamber and at least three channels, each channel extending from the central swirl chamber to the outer diameter of the swirl chamber insert, wherein the ratio of the diameter of the central swirl chamber to the average width of the channels is between about 2.5 and about 3.3, it is deemed a “conventional” pump spray device herein, although it may or may not be in the prior art. Thus, the pump spray device of FIGS. 1 and 2 is used for illustrative purposes only and equivalent pump spray devices known to those of skill in the art, although somewhat different in form or operation from the one described in FIGS. 1 and 2, may incorporate the central swirl chamber or be modified to incorporate the central swirl chamber according to the present invention according to the present invention, as would be apparent to those of skill in the art, and is intended to be embraced within the present invention.

Typical dimensions for the central swirl chamber and channel widths of the swirl chamber insert are shown in Table I, where Pump A is outside the scope of the present invention and included for comparison purposes and Pumps B, C, and D are pump spray devices according to the present invention.

TABLE I

| | Diameter of | | | | Average | |
|---|---|---|---|---|---|---|
| | Central Swirl | Channel Width | | | Channel | |
| | Chamber (mm) | (mm) | | | Width (mm) | Ratio of |
| Pump | d | L1 | L2 | L3 | L_{avg} | D/L_{avg} |
| A | 1.01 | 0.32 | 0.29 | 0.27 | 0.293 | 3.443 |
| B | 1.07 | 0.38 | 0.38 | 0.38 | 0.380 | 2.816 |
| C | 1.02 | 0.36 | 0.35 | 0.36 | 0.357 | 2.860 |
| D | 1.01 | 0.35 | 0.38 | 0.35 | 0.360 | 2.806 |

### Ovality Measurement Protocol

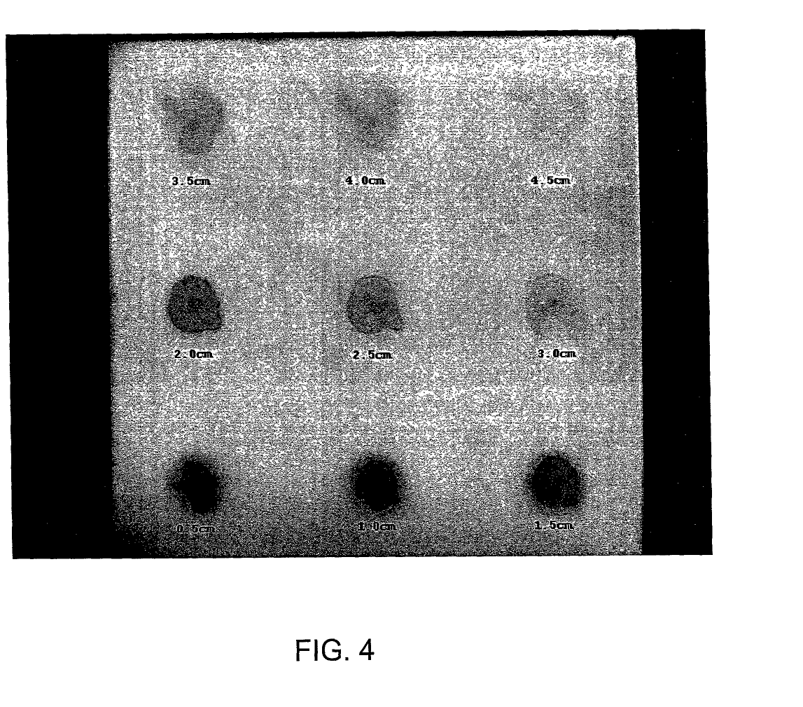

Spray pattern characterizes the spray following impaction on an appropriate target, for example, a thin-layer chromatographic (TLC) plate. Spray pattern is generally determined on a single actuation at several, preferably 3 or more, appropriate distances, e.g., about 0.5 cm to about 6.0 cm, about 0.75 cm to about 5.0 cm, about 1.0 cm to about 4.0 cm, about 1.5 cm to about 3.5 cm, and about 2.0 cm to about 3.0 cm, from the actuator to the target. The visualization technique used is specific for fluticasone propionate: the spray pattern is viewed under 254 nm UV light. Clear, legible photographs of photocopies of the spray pattern are obtained. An example of such spray pattern visualization obtained for Pump D of Table I for single sprays at 0.5 cm, 1.0 cm, 1.5 cm, 2.0 cm, 2.5 cm, 3.0 cm, 3.5 cm, 4.0 cm, and 4.5 cm is shown in FIG. 4. The widest (Dₘₐₓ) and shortest (Dₘᵢₙ) diameters are of such sprays are measured. The ovality ratio (Dₘₐₓ/Dₘᵢₙ) is calculated for each spray. The average ovality ratio values of approaching 1.0 represents a circular pattern, whereas the ovality ratio of substantially greater than 1.5 represent an elliptical or irregular pattern.

### Ovality Measurement Experiments

The fluticasone lots were prepared according to the invention and tested for the spray pattern according to the above protocol.

Table II show fluticasone propionate lot numbers, number of bottles tested, and spray pattern results of pump spray devices according to the instant invention.

TABLE II

| | | Number of | | Range |
|---|---|---|---|---|
| Lot | Number of | Sprays | Average | Ovality Ratio |
| Number | Bottles Tested | per Bottle | Ovality Ratio | (Min-Max) |
| 009014C | 10 | 3 | 1.25 | (1.06-1.57) |
| 009015C | 10 | 3 | 1.22 | (1.03-1.54) |
| 009016C | 10 | 3 | 1.20 | (1.05-1.58) |
| 009014D | 10 | 3 | 1.16 | (1.00-1.36) |
| 009015D | 10 | 3 | 1.20 | (1.05-1.66) |
| 009016D | 10 | 3 | 1.15 | (1.05-1.28) |

we claim:

1. An aqueous pharmaceutical formulation suitable for use in a pump spray device, comprising:
(a) fluticasone propionate;
(b) an antimicrobial preservative;
(c) a surfactant;
(d) a tonicity agent; and
(e) a suspending agent.

2. The aqueous pharmaceutical formulation according to claim 1, wherein the antimicrobial preservative is selected from the group consisting of: benzalkonium chloride, methylparaben, sodium benzoate, benzoic acid, phenyl ethyl alcohol, and mixtures thereof.

3. The aqueous pharmaceutical formulation according to claim 1, wherein the surfactant is selected from the group consisting of: Polysorbate 80 NF, polyoxyethylene 20 sorbitan monolaurate, polyoxyethylene (4) sorbitan monolaurate, polyoxyethylene 20 sorbitan monopalmitate, polyoxyethylene 20 sorbitan monostearate, polyoxyethylene (4) sorbitan monostearate, polyoxyethylene 20 sorbitan tristearate, polyoxyethylene (5) sorbitan monooleate, polyoxyethylene 20 sorbitan trioleate, polyoxyethylene 20 sorbitan monoisostearate, sorbitan monooleate, sorbitan monolaurate, sorbitan monopalmitate, sorbitan monostearate, sorbitan trilaurate, sorbitan trioleate, sorbitan tristearate, and mixtures thereof.

4. The aqueous pharmaceutical formulation according to claim 1, wherein the tonicity agent is selected from the group consisting of: dextrose, lactose, sodium chloride, and mixtures thereof.

5. The aqueous pharmaceutical formulation according to claim 1, wherein the suspending agent is selected from the group consisting of: microcrystalline cellulose, carboxymethylcellulose sodium NF, polyacrylic acid, magnesium aluminum silicate, xanthan gum, and mixtures thereof.

6. The aqueous pharmaceutical formulation according to claim 1, comprising:
(a) about 0.03% to about 0.07% (w/w) of fluticasone propionate;
(b) about 0.05% to about 0.50% (w/w) of the antimicrobial preservative;
(c) about 0.001% to about 0.050% (w/w) of the surfactant;
(d) about 1.0% to about 10.0% (w/w) of the tonicity agent; and
(e) about 0.5% to about 5.0% (w/w) of a suspending agent.

7. The aqueous pharmaceutical formulation according to claim 1, comprising:
(a) about 0.04% to about 0.06% (w/w) of fluticasone propionate;
(b) about 0.08% to about 0.40% (w/w) of the antimicrobial preservative;
(c) about 0.004% to about 0.030% (w/w) of the surfactant;
(d) about 3.0% to about 7.0% (w/w) of the tonicity agent; and
(e) about 1.0% to about 3.0% (w/w) of a suspending agent.

8. The aqueous pharmaceutical formulation according to claim 1, comprising:
(a) about 0.04% to about 0.06% (w/w) of fluticasone propionate;
(b) about 0.01% to about 0.40% (w/w) of phenylethyl alcohol and benzalkonium chloride;
(c) about 0.004% to about 0.030% (w/w) of Polysorbate 80 NF;
(d) about 3.0% to about 7.0% (w/w) of dextrose; and
(e) about 1.0% to about 3.0% (w/w) of microcrystalline cellulose and carboxymethylcellulose sodium NF.

9. A method of administering a pharmaceutical formulation to a host in need of such treatment, comprising spraying the aqueous pharmaceutical formulation according to claim 1 using a nasal pump spray device, wherein the average ovality ratio of the spray produced is between about 1.0 and about 1.7.

10. The method of claim 9, wherein the average ovality ratio of the spray produced is between about 1.1 and about 1.5.

11. The method according to claim 10, wherein the average ovality ratio of the spray produced is between about 1.1 and about 1.3.

12. In a pump spray device for a pharmaceutical formulation, the pump spray device comprising an actuator and a pump, wherein the improvement comprises:
a swirl chamber insert with a central swirl chamber and at least three channels, each channel extending from the central swirl chamber to the outer diameter of the swirl chamber insert,
wherein the ratio of the diameter of the central swirl chamber to the average width of the channels is between about 2.5 and about 3.3.

13. The pump spray device of claim 12, wherein the ratio of the diameter of the central swirl chamber to the average width of the channels is between about 2.6 and about 3.1.

14. The pump spray device of claim 13, wherein the ratio of the diameter of the central swirl chamber to the average width of the channels is between about 2.7 and about 3.0.

15. The pump spray device of claim 14, wherein the ratio of the diameter of the central swirl chamber to the average width of the channels is between about 2.8 and about 3.0.

16. Fluticasone propionate having a surface area (BET) in the range of about 7 M²/g to about 12 m ²/g.

17. Fluticasone propionate according to claim 16, wherein the surface area (BET) is in the range of about 8 m ²/g to 1 0 M²/g.

18. Fluticasone propionate according to claim 17, wherein the surface area (BET) is in the range of about 9 m²/g to 10 m²/g.
